(19) United States
(12) Patent Application Publication
(10) Pub. No.: US 2023/0338981 A1
(43) Pub. Date: Oct. 26, 2023

(54) ATOMIZATION EQUIPMENT

(71) Applicant: CHENG-CHUNG SUI, Taichung City (TW)

(72) Inventor: CHENG-CHUNG SUI, Taichung City (TW)

(21) Appl. No.: 18/135,184

(22) Filed: Apr. 16, 2023

(30) Foreign Application Priority Data
Apr. 21, 2022 (TW) 111204091

(51) Int. Cl. B05B 17/06; B05B 7/00

(52) U.S. Cl. CPC B05B 17/0615; B05B 17/0676; B05B 7/0012; F24F 6/12

(57) ABSTRACT

The present invention provides an atomization equipment which comprises a lid and a shell. The lid is configured to have multiple gas chambers, and the lid engages with the shell. Thereafter, the shell comprises a plurality of windows, a tank, at least one vibrator, a liquid input, a liquid stopper and at least one blower. The multiple gas chambers match and communicate with the plurality of windows, and the at least one vibrator are configured at bottom of the tank. On the other hand, the liquid input is configured on the side wall of the tank, and the liquid stopper be detachably closed the liquid input. At last, the at least one blower communicates with the tank.

### TECHNICAL FIELD

The present invention provides an atomization equipment. Specifically, the atomization equipment may add liquid automatically and reduce the waste of atomization.

### BACKGROUND OF RELATED ARTS

For atomization technology, it has always been closely related to human life. For example, many people need to cool down both indoor and outdoor environments in summer. Especially for outdoor environments, it is common method for cooling with atomized water.

In addition to the needs of water mist for cooling, an alternative use which is quite important is directed to disinfection and sterilization in personal daily life. Especially, if there is a simple device that can automatically generate disinfectant water mist while specific infectious diseases are rapidly spread during certain period, it will be quite convenient.

However, the commercial atomization equipment often has several disadvantages. The first drawback is that the atomized water mist is not uniform enough that may cause a lot of water mist to be released directly in the form of liquid without becoming mist, wasting a lot of liquid. In addition, the container carrying the liquid cannot automatically replenish the liquid, resulting in the device needs to be replenished manually and frequently, which is quite inconvenient to use.

Therefore, there is an urgent need for an atomization equipment that can automatically replenish liquid stably and release water mist in a uniform and non-wasting liquid form.

### SUMMARY

In order to solve the problems mentioned in the prior art, the present invention provides an atomization equipment which comprise a lid and a shell.

The lid is configured with a plurality of gas chambers, and the shell engages with the lid. The shell comprises a plurality of windows, a tank, at least one vibrator, a liquid input, a liquid stopper and at least one blower. The windows match and communicate with the gas chambers, and at least one vibrator is configured at bottom of the tank. The liquid input is configured on the side wall of the tank. The liquid stopper be detachably closed the liquid input. At least one blower communicates with the tank.

The above-mentioned descriptions are only preferred embodiments of the present invention and are not intended to limit the scope of implementation of the present invention. Therefore, all the shapes, structures, features, and spirits described in the scope of the patent application of the present invention shall be regarded as equivalent to the changes and modifications per se, and be included in the scope of the patent application of the present invention.

### BRIEF DESCRIPTION OF THE DRAWINGS

FIG. 1 is a cross section structure diagram of the atomization equipment of the present invention.

FIG. 2 is a lid structure schematic diagram of the atomization equipment of the present invention.

FIG. 3 is a schematic diagram of the mist spraying of the atomization equipment of the present invention.

FIG. 4 is a schematic diagram of the liquid inputting of the atomization equipment of the present invention.

FIG. 5 is a schematic diagram of the water container operation of the atomization equipment of the present invention.

FIG. 6 is a schematic diagram of the water container liquid refilling of the atomization equipment of the present invention.

### DETAILED DESCRIPTION OF THE INVENTION

To make the description of the present disclosure more detailed and complete, the following description provides an illustrative description for the implementation and specific embodiments of the present invention. However, the following description is not the only form of implementing or using specific embodiments of the invention. In these paragraphs, the features of various specific embodiments are covered as well as the method steps and sequences for constructing and operating these specific embodiments. However, the other embodiments may also be utilized to achieve the same or equivalent function and sequence of steps.

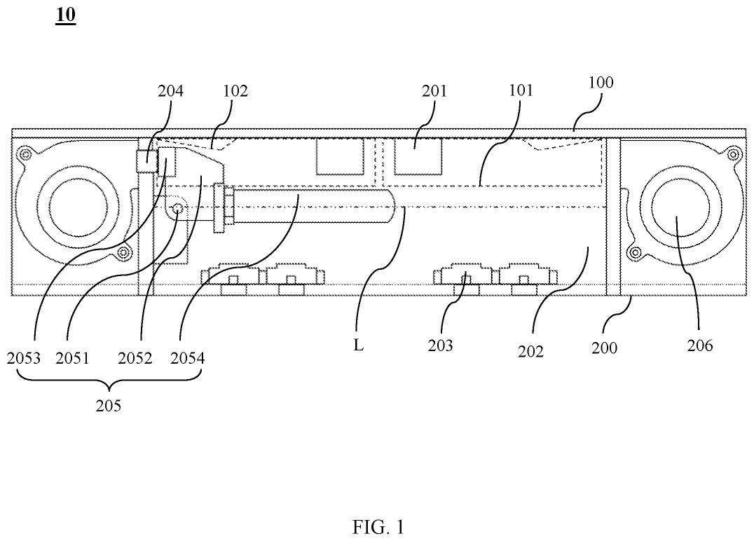

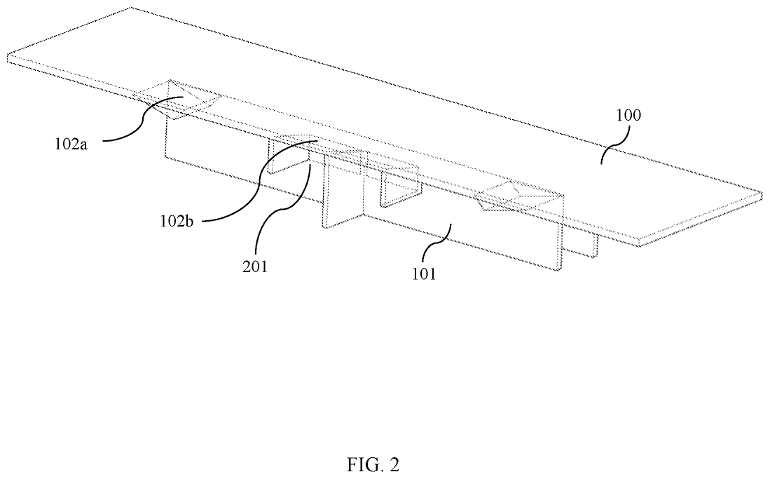

As shown in FIG. 1 and FIG. 2. FIG. 1 is a cross section structure diagram of the atomization equipment of the present invention. FIG. 2 is a lid structure schematic diagram of the atomization equipment of the present invention.

Specifically, this embodiment of the present invention provides an atomization equipment 10. The shape of the atomization equipment 10 is a square cube. The atomization equipment 10 of this embodiment comprises the lid 100 and the shell 200. The shell 200 comprises a plurality of window 201, tank 202, vibrator 203, liquid input 204, liquid stopper 205, and blower 206. Although we see two blowers in the cross section. Actually, there are four blowers in this embodiment because blowers are configured on the side of tank 202 in pairs. The blower 206 is connected to the tank 202 by aligning. In addition, two blowers on the left side are configured to be clamped to the liquid input 204, and the liquid input 204 is configured on the side wall of the tank 202.

The blower 206 of this embodiment may is a gas pump. The liquid input 204 is main used for adding liquid. Specifically, the liquid input 204 of this embodiment further connect to the liquid supply source. The liquid supply source can be a spare liquid bottles, cans, or barrels, etc. and can be a connecting pipe with outer supply liquid.

In the embodiment, the lid 100 and shell 200 are made of acrylic acid, and more precisely transparent acrylic acid. In this embodiment, we can easily realize the inner operation condition of the atomization equipment 10 due to the transparency of acrylic acid. The user can quickly find the malfunction part of the atomization equipment 10.

In the embodiment, there are a plurality of gas chambers 101 configured on the lid 100, and the shell 200 engages with the lid 100. As shown in FIG. 2, the space of the gas chambers 101 of this embodiment is form of block plates is configured on the bottom of the lid 100. There are four gas chambers in this embodiment. FIG. 2 only show a side of the lid 100, so there are only two gas chambers 101 shown in this figure.

Specifically, the gas chamber 101 of this embodiment also includes the inclined plate 102a and the inclined plate 102b. The inclined plate 102a is configured on a way of the air outlet. The inclined plate 102b is configured on the top of a channel between gas chamber 101 and window 201. Specifically, inclined plate 102a and inclined plate 102b can assist condensation liquid generated on the channel which slid down into tank 202 by gravity which can reduce greatly waste liquid from condensed and not atomizing.

As shown in FIG. 1, the vibrator 203 is configured on the bottom of the tank 202. Specifically, the vibrator 203 of this embodiment is an ultrasonic vibrator. Hence, the vibrator 203 of this embodiment further connects at least one mechanical-electric driving device. The microcontroller or microprocessor of the mechanical-electric driving device can atomize a variety kind of liquids with different molecular mass by ultrasonic vibrator. The ultrasonic vibrator can provide suitable vibrating frequencies for this liquid.

Moreover, the liquid stopper 205 of this embodiment be detachably closed the liquid input 204. The liquid stopper 205 of this embodiment is constructed by pivot joint 2051, connecting unit 2052, sealing plate 2053, and float 2054. The quantity of float 2054 could be changed by needs.

The pivot joint 2051 of this embodiment is configured on the lower of the liquid input 204. The pivot joint 2051 could be a pivot pin configured on the plates. The connecting unit 2052 is connected rotationally to the pivot joint 2051. In this embodiment, the connecting unit 2052 is formed by two side plates. Two side plates clamp the pivot joint 2051 which is the axis of the rotation that can make the connecting unit 2052 can be rotated by the center (such as pivot joint 2051) for a certain distance.

Therefore, the sealing plate 2053 of this embodiment is configured on the connecting unit 2052 and is used for closing the liquid input 204. The sealing plate 2053 can be made of waterproof materials such as rubber or silicone because sealing plate 2053 of this embodiment has to impact the liquid input 204 and achieve sealing from water.

Finally, the float 2054 of this embodiment is fixed to the side of the connecting unit 2052, and the sealing plate 2053 is fixed to the other side of the connecting unit 2052. When the liquid level L of tank 202 changes, the position of float 2054 will be changed which makes sealing plate 205 rotate, and liquid input 204 will be opened.

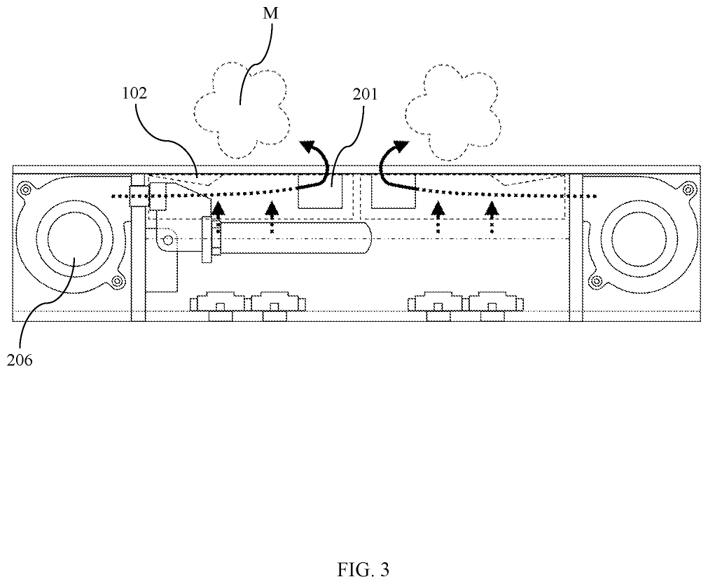

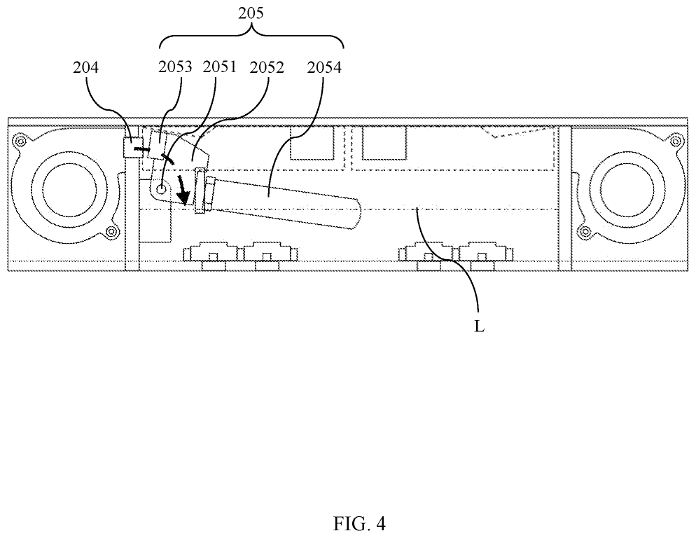

As shown in FIG. 3 and FIG. 4, FIG. 3 is a schematic diagram of the mist spraying of the atomization equipment of the present invention. FIG. 4 is a schematic diagram of the liquid inputting of the atomization equipment of the present invention.

As shown in FIG. 3, when this embodiment has to generate mist M, vibrator 203 will vibrate liquid in tank 202 to be an atomized liquid. When the atomized liquid raises up to gas chamber 101, the atomized liquid will be blown to window 201 along the gas chamber 101 by blower 206, and atomized liquid will spray mist when going out of window 201 such as formed mist M.

As time passes, the liquid is changed to mist M continuously which makes the position of liquid level L drop. Dropped liquid level L drives float 2054 will be rotated which makes connecting unit 2052 and sealing plate 2053 be rotated in a clockwise direction. As sealing plate 2053 rotates to leave away liquid input 204. Hence, liquid input 204 will be opened without blocking from sealing plate 2053, and liquid input 204 refill new liquid to tank 202 as dotted allow shown in FIG. 4, and refilling makes liquid level L raise.

After liquid level L is raised to a default level, float 2054 will be rotated to drive sealing plate 2053 rotate in counterclockwise direction by connecting to connecting unit 2052 and the rotation makes sealing plate 2053 close liquid input 204. The design structure in this embodiment makes the tank 202 be refilled liquid automatically.

In the embodiment, the microcontroller and microprocessor of the mechanical-electric driving device further can connect to an infrared sensor or optical sensor. When the sensor detecting a specific object or fit the specific condition, vibrator 203 will be turn on to atomize.

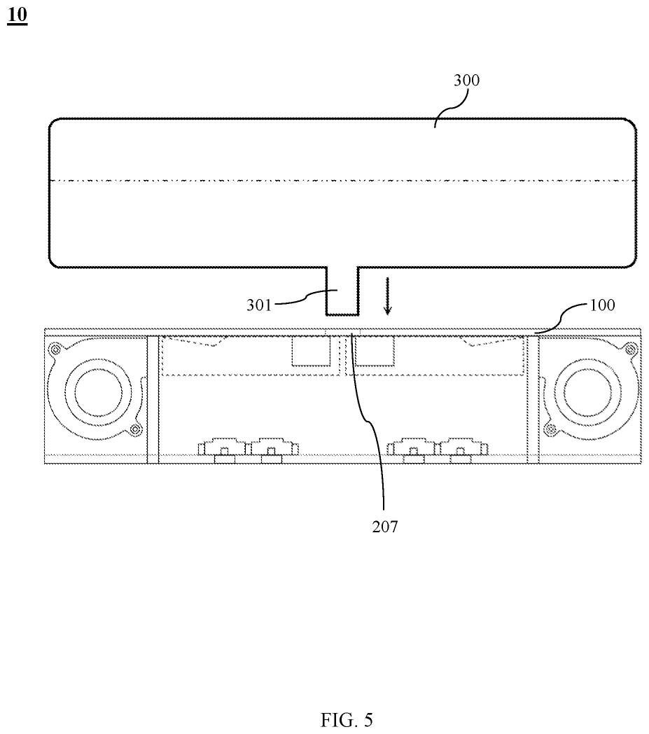

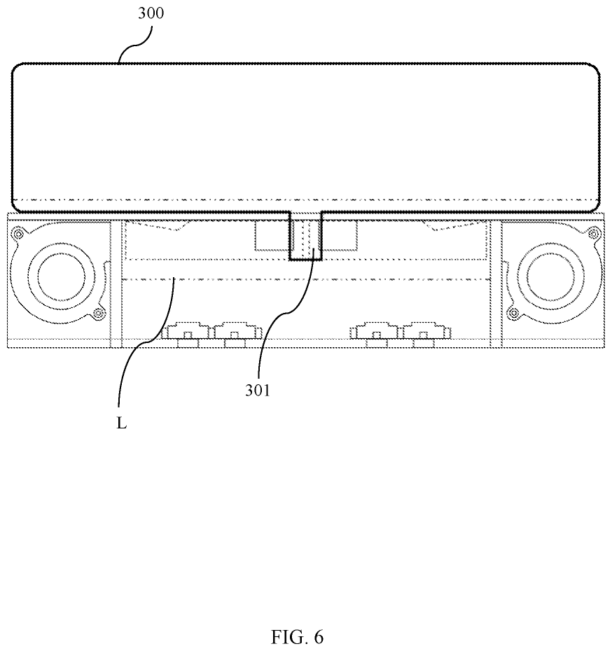

Further, the present invention provides a function for adding liquid to the liquid level manually. As shown in FIG. 5 and FIG. 6, FIG. 5 is a schematic diagram of the water container operation of the atomization equipment of the present invention. FIG. 6 is a schematic diagram of the water container liquid refilling of the atomization equipment of the present invention.

As shown in FIG. 5, in this embodiment, lid 100 further comprises refilled water inlet 207. Specifically, refilled water inlet 207 can further connect to water container 300. In the embodiment, liquid can be refilled into the water container 300 manually.

The water container 300 of this embodiment further comprises a plugged-inlet 301 protruding from the water container 300. The plugged-inlet 301 is a tube that connects to the water container 300 and whose outer shape can be a cylinder, rectangular prism, polygonal prism, etc. Hence, the shapes of the refilled water inlet 207 are designed for matching to the shape of the plugged inlet 301 which makes them paired with each other by the female and male connecting way.

Accordingly, the user can take any leakless approach to fill the water container 300 through the plugged inlet 301 (i.e. the water container 300 is placed with the plugged inlet 301 facing upwards). After the water container 300 is full, the user places the water container 300 upsides down, and makes the plugged inlet 301 align to the refilled water inlet 207, and then the water container 300 will fill into the atomization equipment automatically. The aligned direction is like the arrow shown in FIG. 5.

Specifically, the plugged inlet 301 can be designed to include a valve whose window way is turning to a specific angle in order to avoid leaking when the water container 300 upsides down. Refilled water inlet 207 also can be a valve. The user can be refilled by the design of plugged inlet 301 on the water container 300.

As shown in FIG. 6, when plugged inlet 301 of water container 300 be plugged into atomization equipment 10 via refilled water inlet 207, liquid of water container 300 will flow from water container 300 into atomization equipment 10 via plugged inlet 301 that makes liquid level L of atomization equipment 10 raise.

The above-mentioned descriptions are only preferred embodiments of the present invention and are not intended to limit the scope of implementation of the present invention. Therefore, all the shapes, structures, features, and spirits described in the scope of the patent application of the present invention shall be regarded as equivalent to the changes and modifications per se, and be included in the scope of the patent application of the present invention.

What is claimed is:

1. An atomization equipment, comprises:
a lid, configured with a plurality of gas chambers;
a shell engaging with the lid, wherein the shell comprises:
a plurality of window, matched and communicated with the plurality of gas chambers;
a tank;
at least one vibrator, configured at bottom of the tank;
a liquid input, configured on the side wall of the tank;
a liquid stopper detachably closing the liquid input; and
at least one blower communicating with the tank.

2. The atomization equipment as claimed in claim 1, wherein each of the gas chamber comprises at least one inclined plate.

3. The atomization equipment as claimed in claim 1, wherein a number of the at least one blower is four, and the four blowers are arranged in pairs on both sides of the tank.

4. The atomization equipment as claimed in claim 1, wherein the atomization equipment is square cube shaped.

5. The atomization equipment as claimed in claim 1, wherein the at least one vibrator is an ultrasonic vibrator.

6. The atomization equipment as claimed in claim 1, wherein the liquid stopper comprises:
a pivot joint, configured on bottom of the liquid input;
a connecting unit, rotatably connected to the pivot joint;
a sealing plate, configured on the connecting unit, and the sealing plate is used for closing the liquid input; and
at least one float, connected to the connecting unit;
wherein once a liquid level of the tank drops, the connecting unit with the sealing plate will be rotated by actuating of the float and then open the liquid input.

7. The atomization equipment as claimed in claim 6, wherein the connecting unit is formed by two side plates.

8. The atomization equipment as claimed in claim 1, wherein the liquid input further connects to a liquid supply source.

9. The atomization equipment as claimed in claim 1, wherein the at least one blower further connects to at least one mechanical-electric driving device.

10. The atomization equipment as claimed in claim 1, wherein a refilled water inlet is further configured on the lid.

11. The atomization equipment as claimed in claim 10, wherein the refilled water inlet further detachably connects to a water container.

12. The atomization equipment as claimed in claim 11, wherein a plugged inlet is configured on the water container, and shapes of the plugged inlet and the refilled water inlet are matched with each other.
